(19) United States
(12) Patent Application Publication
(10) Pub. No.: US 2005/0136533 A1
(43) Pub. Date: Jun. 23, 2005

(54) Biochip substrate holding method and biochip-reader

(75) Inventors: Yumiko Sugiyama, Tokyo (JP); Takeo Tanaami, Tokyo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: YOKOGAWA ELECTRIC CORPORATION, Tokyo (JP)

(21) Appl. No.: 10/928,184

(22) Filed: Aug. 30, 2004

(30) Foreign Application Priority Data
Dec. 22, 2003 (JP) 2003-424539

(51) Int. Cl. C12Q 1/68; G06K 9/00; C12M 1/34

(52) U.S. Cl. 435/287.2; 382/128

(57) ABSTRACT

In measurement of a biochip whose substrate is held on a base and samples are spotted onto the sites in an array on the substrate, a biochip substrate holding method, which does not generate deviation of site positions between the arrangement of multiple samples on the biochip and mounting of the biochip onto the biochip-reader and does not need position aligning, can be provided by holding the biochip substrate with equivalent holding mechanisms in spotting and in measurement respectively. A biochip-reader using the above mentioned method can also be provided.

### BACKGROUND OF THE INVENTION

1. Field of the Invention

The present invention relates to a biochip substrate holding method and a biochip-reader using the above mentioned method, and in more detail, to a holding method in which positions of biochip sites can be arranged in a good repeatability and a biochip-reader using the above mentioned method.

2. Description of the Prior Art

There are conventional biochip-readers which are configured to read fluorescence generated from a sample by irradiating exciting light such as laser light onto the sample in each biochip site (for example, refer to Patent Document 1).

These conventional biochip-readers include scanning types (scan type) that use a microlens array in which a plurality of microlenses is arranged and the irradiating beam is scanned after passing through the microlenses, or non-scanning types (scan-less type) that use no scanning, or those that do not use a microlens-array.

In all of these biochip-readers, a substrate used for biochips (hereinafter called the biochip substrate or simply the substrate) is normally fixed to a base using a sample holder adopted for microscopes or its equivalent. FIG. 1 shows an example of the above sample holder mechanism. The sample holder is (as shown in FIG. 1) equipped with glass slide support 2 and pivoting arm 4 which is mounted so as to enable it to pivot via coupling 3 on base 1 and is used to press end faces 5a and 5b of the two sides of rectangular glass slide (equivalent to the biochip substrate) 5, adjacent to each other, to glass slide support 2 using the action of a spiral spring (not shown in the drawing) provided in coupling 3 (for example, refer to Patent Document 2).

[Patent Document 1]

Gazette for Japanese Laid-open Patent Application No. 2003-028799

[Patent Document 2]

Gazette for Japanese Laid-open Patent Application No. 10-39230

However, conventional sample holders have the following disadvantages:
(1) Because the positioning mechanism of sample holders is insufficient in biochip mounting and dismounting, positions of biochip sites deviate in directions of x, y, and/or z (the directions of x and y are those orthogonal to the optical axis and the direction of z is the direction of the optical axis itself). This necessitates position-locating after mounting.
(2) In the case of a scan-less type, positions of microlenses deviate from positions of sites and thus position-aligning becomes necessary.
(3) Even if the sample holder is not a scan-less type, deviation of positions must be corrected using pattern recognition or the like.
(4) Since, for biochip substrates, accuracy in their external dimensions is not very precise that their sides are not straight and the adjacent two sides are also not perpendicular to each other, positioning using glass slide support 2 of the above mentioned sample holder and pivoting arm 4 is not exact, and its repeatability is low, introducing no exact positioning.
(5) Since the biochip fixing scheme when samples are spotted to each site of a biochip is different from the biochip fixing scheme used in the sample holder, in the case of a scan-less type, deviations (in the directions of x, y, and θ) are generated between sites 6 and corresponding light beam irradiation positions 7, as shown in FIG. 2(a). In addition, θ shows the angle formed in the surface of the biochip substrate 14, which is orthogonal of the optical axis. Each site and corresponding light beam irradiation position must coincide with the other as shown in FIG. 2(b).

### SUMMARY OF THE INVENTION

The objective of the present invention is to solve the above problems and so to offer a biochip substrate holding method which does not cause deviation of site positions during measurement, and thus does not require position-aligning by making holding mechanisms of biochip substrate equal both in arranging multiple samples on a biochip and in mounting a biochip to a biochip-reader, and to offer a biochip-reader using the above mentioned method.

### BRIEF DESCRIPTION OF DRAWINGS

FIG. 1 is a configuration drawing indicating an example of conventional sample holders.

FIG. 2 is a drawing showing the relationship between site positions and corresponding light beam irradiation positions on a biochip substrate.

FIG. 3 is a configuration drawing indicating an embodiment of the substrate holding mechanism for achieving the biochip holding method concerning the present invention.

FIG. 4 gives two bird's-eye views of a biochip substrate holding mechanism of the present invention.

FIG. 5 is a drawing indicating another embodiment of a biochip substrate holding mechanism of the present invention.

FIG. 6 is a drawing indicating further another embodiment of a biochip substrate holding mechanism of the present invention.

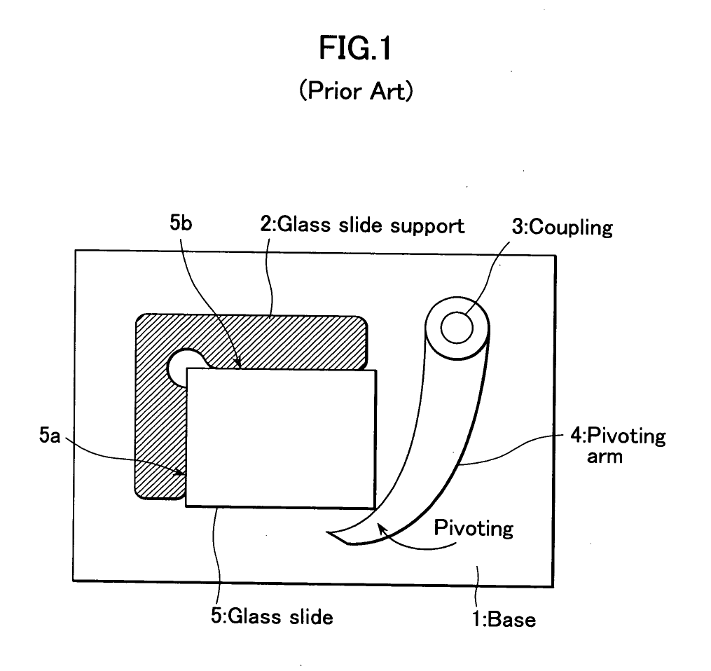

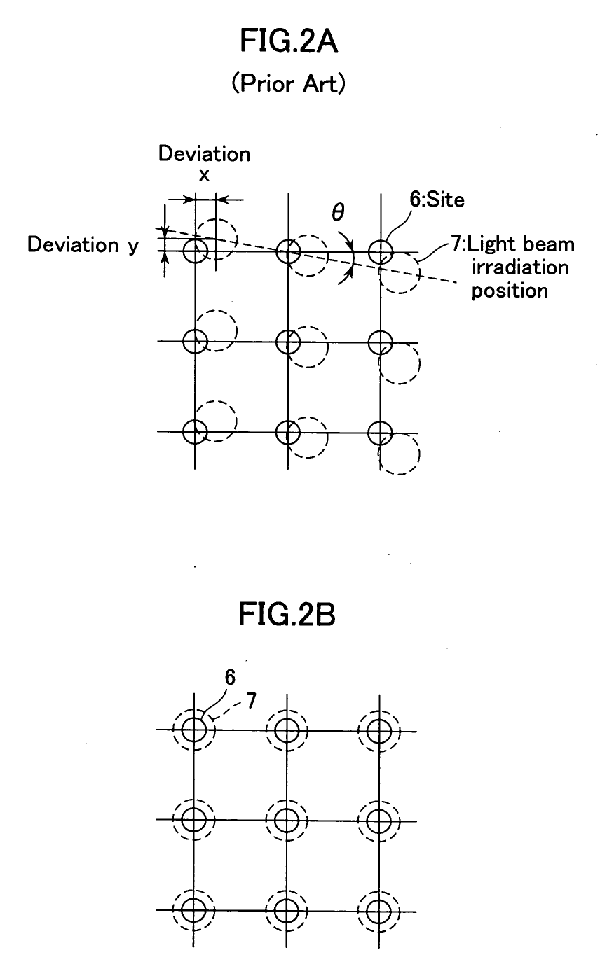

### DESCRIPTION OF THE PREFERRED EMBODIMENTS

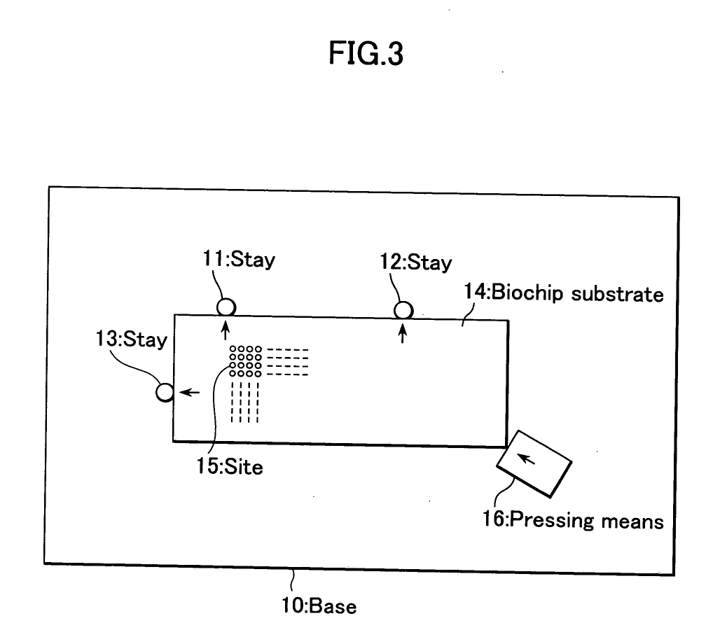

The present invention will be described below in detail using drawings. FIG. 3 is a configuration drawing indicating an embodiment of the substrate holding mechanism for achieving the biochip holding method concerning the present invention.

In FIG. 3, numeral 10 denotes a base, numerals 11, 12 and 13 denote stays attached to base 10, numeral 14 a biochip substrate (hereafter simply called “substrate”) on which multiple samples are arranged in an array, numeral 15 denotes sites on substrate 14, and numeral 16 a pressing means.

Stays 11 to 13 are fixed to base 10 so that the side faces of two adjacent sides of substrate 14 mounted onto base 1 touch these stays. These stays are formed with round columns or cylinders (for example, pins) and configure point contacts with side faces of substrate 14.

Pressing means 16 applies a pressing force in an oblique direction towards the touching sides from the corner where two sides, not being the touching sides, cross each other, when substrate 14 is touched to the three stays.

In such a construction, if two adjacent sides of substrate 14 are pressed to stays 11 to 13, substrate 14 is always mounted on the predetermined position of base 10 with good repeatability, without being affected by the bend of the sides or the angle between two sides because substrate 14 contacts with these three points only.

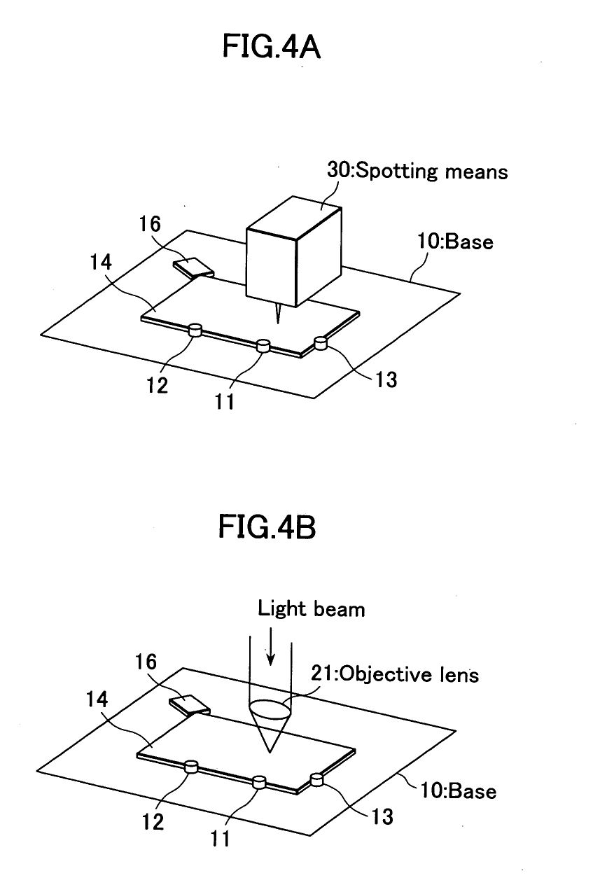

In this case, as shown in FIG. 4, the substrate is held using essentially the same three-point contact structure either in spotting of biochip samples (FIG. 4(a)) or during measurements with the reader (FIG. 4(b)). Employing the above structure eliminates the generation of deviation in site positions when measurement is made.

Further, the present invention is not restricted to the above embodiment but may be embodied in other specific forms, changes, and versions without departing from the true spirit thereof.

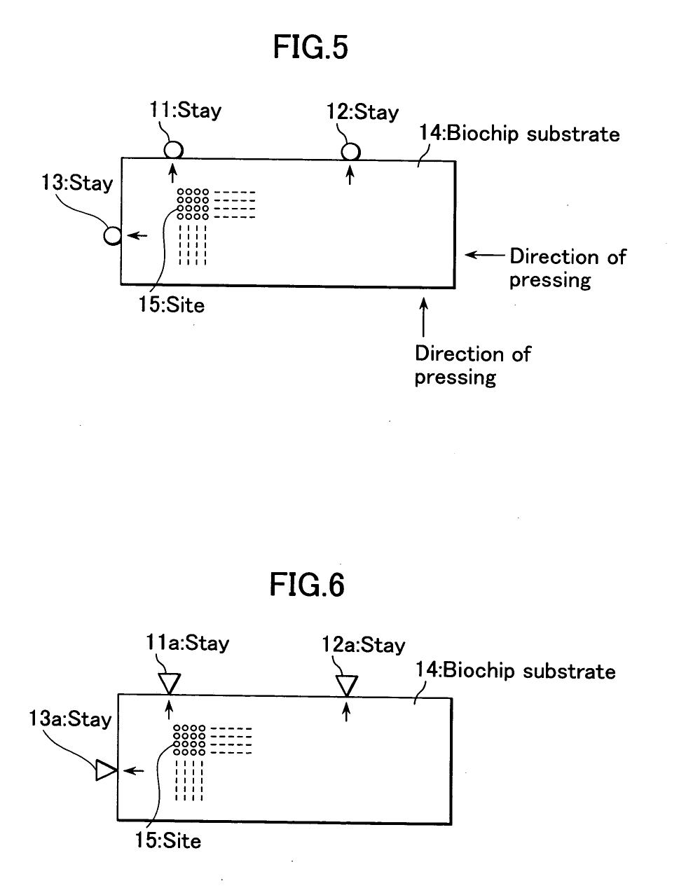

For example, it is also acceptable to apply the pressing force by pressing means 16 in two directions perpendicular to each side of two sides orthogonal to each other as shown in FIG. 5, not the oblique direction as described in the above embodiment.

Further, the external shape of stays 11 to 13 is not limited to a round type but may be polygonal, for example, triangular as shown in FIG. 6. However, their edges must be touching in point contacts with the biochips.

In addition, DNA, RNA, proteins, and bio-metabolites (low-molecular materials in living bodies other than protein) and the like are used as samples.

Further, spotting of samples to the sites of a biochip can be carried out using pin, ink-jet, electrostatic adsorption and the like by holding a substrate with the substrate holding mechanism of the present invention.

Furthermore, a substrate which is the object of holding may be a glass slide or a cartridge.

As apparent from the above description, the present invention has the following effects:
(1) Positional deviation during measurement using a reader can be minimized by making the biochip substrate holding mechanism of the reader agree with the holding mechanism when samples are spotted onto a biochip substrate.
(2) It is not necessary to perform adjustment of site position-aligning and pattern matching based on images required in conventional readers.
(3) Since the biochip substrate is supported at three points, good supporting reproducibility can be easily obtained without being affected by the linearity of substrate sides and orthogonality of two sides, or the like.
(4) Glass slides or synthetic-resin or cartridges can be used as biochip substrates.

1. A biochip substrate holding method, in which biochip substrates are held, when measuring a biochip obtained by holding a biochip substrate on a base and spotting samples onto the sites arranged in an array on that biochip substrate, by equivalent holding mechanisms in both said spotting and said measurement respectively.

2. A biochip substrate holding method in accordance with claim 1, wherein said biochip substrate is held by touching the adjacent two sides of said biochip substrate to three stays, that is, supporting said substrate in a three-point contact, and
by applying a pressing force to said substrate from the opposite corner where the other two sides of said substrate cross each other or
by applying a pressing forces in two directions perpendicular to each of said two sides, in said biochip substrate holding mechanism.

3. A biochip substrate holding method in accordance with claim 1, wherein said spotting is performed by a pin, ink-jet or electrostatic adsorption.

4. A biochip substrate holding method in accordance with claim 2, wherein said spotting is performed by a pin, ink-jet or electrostatic adsorption.

5. A biochip-reader which is constructed to use a biochip, on which samples are arranged in the sites located in an array respectively, as a measuring object, to irradiate samples with a light beam, and to read reflected light from the samples;
further configured so that the positions of said sites agree with corresponding light beam irradiation positions by making a biochip substrate holding mechanism in a biochip-reader essentially agree with said biochip substrate holding mechanism in arranging multiple samples on said biochip.

6. A biochip-reader in accordance with claim 5, wherein said biochip substrate holding mechanism is a three-point holding mechanism which holds said biochip substrate by supporting said substrate in a three-point contact by touching the adjacent two sides of said biochip substrate to three stays fixed to the surface of the base and by applying a pressing force from the opposite corner where the other two sides of said biochip substrate cross each other.

7. A biochip-reader in accordance with claim 5, wherein said samples include DNA or RNA or proteins or glyco-chains or bio-metabolites.

8. A biochip-reader in accordance with claim 6, wherein said samples include DNA or RNA or proteins or glyco-chains or bio-metabolites.

9. A biochip-reader in accordance with claim 5, wherein the pressing force applied to hold said biochip substrate is an oblique one-directional pressing force from the corner where said two sides cross each other or pressing forces in two directions perpendicular to each of said two sides respectively.

10. A biochip-reader in accordance with claim 6, wherein the pressing force applied to hold said biochip substrate is an oblique one-directional pressing force from the corner where said two sides cross each other or pressing forces in two directions perpendicular to each of said two sides respectively.

11. A biochip-reader in accordance with claim 7, wherein the pressing force applied to hold said biochip substrate is an oblique one-directional pressing force from the corner where said two sides cross each other or pressing forces in two directions perpendicular to each of said two sides respectively.

12. A biochip reader in accordance with claim 8, wherein the pressing force applied to hold said biochip substrate is an oblique one-directional pressing force from the corner where said two sides cross each other or pressing forces in two directions perpendicular to each of said two sides respectively.

13. A biochip-reader in accordance with claim 5, wherein said stays have a round shape or polygonal external shape and are formed to contact with said biochip substrate in the point contact respectively.

14. A biochip-reader in accordance with claim 6, wherein said stays have a round shape or polygonal external shape and are formed to contact with said biochip substrate in the point contact respectively.

15. A biochip-reader in accordance with claim 7, wherein said stays have a round shape or polygonal external shape and are formed to contact with said biochip substrate in the point contact respectively.

16. A biochip-reader in accordance with claim 8, wherein said stays have a round shape or polygonal external shape and are formed to contact with said biochip substrate in the point contact respectively.

17. A biochip-reader in accordance with claim 9, wherein said stays have a round shape or polygonal external shape and are formed to contact with said biochip substrate in the point contact respectively.

18. A biochip-reader in accordance with claim 10, wherein said stays have a round shape or polygonal external shape and are formed to contact with said biochip substrate in the point contact respectively.

19. A biochip-reader in accordance with claim 11, wherein said stays have a round shape or polygonal external shape and are formed to contact with said biochip substrate in the point contact respectively.

20. A biochip-reader in accordance with claim 12, wherein said stays have a round shape or polygonal external shape and are formed to contact with said biochip substrate in the point contact respectively.
